SEQUENCE LISTING (13 sequences)

SEQ ID NO: 1 - protein, 120 aa, synthetic construct
EVQLVESGGGLVQPGGSLRLSCAASGFPFSNYAMSWYRQAPGKERELVANIGGPSGYSTYYKEAVKGRFTISRDNAKTTVYLHMNSLKPDDTAVYFCKAVARIGVPQEYWGQGTQVTVSS

SEQ ID NO: 2 - protein, 365 aa, synthetic construct
MALPVTALLLPLALLLHAARPEVQLVESGGGLVQPGGSLRLSCAASGFPFSNYAMSWYRQAPGKERELVANIGGPSGYSTYYKEAVKGRFTISRDNAKTTVYLHMNSLKPDDTAVYFCKAVARIGVPQEYWGQGTQVTVSSTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 1095 bp, synthetic construct
  source 1..1095: mol_type = other DNA
atggccctgccagtgaccgccctcctgctgcctctggctctgctgctgcacgccgccagacccgaggtgcagctcgtggagagcggcggcgggctggtgcagcctggaggctccctgcggctgagctgcgccgcaagcggttttcccttctccaattatgcaatgagctggtatcggcaggctccaggcaaagagagagaactggtggccaacatcggcggcccctctggatatagcacctactataaagaggccgtgaagggcagatttaccatcagcagagataacgccaagacaacagtgtacctgcatatgaattctctgaaaccagacgataccgccgtgtacttctgcaaggccgtggcccgcattggagtgccccaggagtactggggccagggcacccaggtgaccgtgtcttccacaaccacacccgcccctagacctccaacccccgcccccactatcgcttcccagcctctgtctctcagacccgaggcctgtaggcccgccgccggcggcgccgtgcacacccgggggctggatttcgcatgtgacatttacatctgggctccactggctggcacatgcggagtgctgctgctgagtctggtgattaccttgtactgcaacaagcggggccgcaagaagctgctgtatattttcaaacagccctttatgcgccctgtgcagaccacccaggaagaggacggctgctcctgcaggttcccagaagaggaagagggagggtgcgagctgagggtgaaattctccagaagcgccgatgcacccgcatatcagcagggacagaaccagctgtacaacgagctgaatctgggacggagagaggagtatgatgtgctggataagcgcaggggcagagaccccgagatgggcggcaaacctagacgcaagaaccctcaggagggcctgtataatgagctgcagaaggataagatggctgaagcctattctgagatcggaatgaagggtgagagaaggcggggcaaagggcacgatggactgtaccagggactgtctactgctactaaggacacctacgacgccctgcatatgcaggccctgcccccccgc

SEQ ID NO: 4 - DNA, 120 bp, synthetic construct
  source 1..120: mol_type = other DNA
atggccctgccagtgaccgccctcctgctgcctctggctctgctgctgcacgccgccagacccgaggtgcagctcgtggagagcggcggcgggctggtgcagcctggaggctccctgcgg

SEQ ID NO: 5 - DNA, 135 bp, synthetic construct
  source 1..135: mol_type = other DNA
acaaccacacccgcccctagacctccaacccccgcccccactatcgcttcccagcctctgtctctcagacccgaggcctgtaggcccgccgccggcggcgccgtgcacacccgggggctggatttcgcatgtgac

SEQ ID NO: 6 - DNA, 75 bp, synthetic construct
  source 1..75: mol_type = other DNA
atttacatctgggctccactggctggcacatgcggagtgctgctgctgagtctggtgattaccttgtactgcaac

SEQ ID NO: 7 - DNA, 126 bp, synthetic construct
  source 1..126: mol_type = other DNA
aagcggggccgcaagaagctgctgtatattttcaaacagccctttatgcgccctgtgcagaccacccaggaagaggacggctgctcctgcaggttcccagaagaggaagagggagggtgcgagctg

SEQ ID NO: 8 - DNA, 336 bp, synthetic construct
  source 1..336: mol_type = other DNA
agggtgaaattctccagaagcgccgatgcacccgcatatcagcagggacagaaccagctgtacaacgagctgaatctgggacggagagaggagtatgatgtgctggataagcgcaggggcagagaccccgagatgggcggcaaacctagacgcaagaaccctcaggagggcctgtataatgagctgcagaaggataagatggctgaagcctattctgagatcggaatgaagggtgagagaaggcggggcaaagggcacgatggactgtaccagggactgtctactgctactaaggacacctacgacgccctgcatatgcaggccctgcccccccgc

SEQ ID NO: 9 - DNA, 7686 bp, synthetic construct
  source 1..7686: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgctgagttaactattctagagtacccgggctaggatccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 10 - DNA, 8274 bp, synthetic construct
  source 1..8274: mol_type = other DNA
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttagtaccgggcccgctctagcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgctgagttaactattctagaatggccctgccagtgaccgccctcctgctgcctctggctctgctgctgcacgccgccagacccgaggtgcagctcgtggagagcggcggcgggctggtgcagcctggaggctccctgcggctgagctgcgccgcaagcggttttcccttctccaattatgcaatgagctggtatcggcaggctccaggcaaagagagagaactggtggccaacatcggcggcccctctggatatagcacctactataaagaggccgtgaagggcagatttaccatcagcagagataacgccaagacaacagtgtacctgcatatgaattctctgaaaccagacgataccgccgtgtacttctgcaaggccgtggcccgcattggagtgccccaggagtactggggccagggcacccaggtgaccgtgtcttccacaaccacacccgcccctagacctccaacccccgcccccactatcgcttcccagcctctgtctctcagacccgaggcctgtaggcccgccgccggcggcgccgtgcacacccgggggctggatttcgcatgtgacatttacatctgggctccactggctggcacatgcggagtgctgctgctgagtctggtgattaccttgtactgcaacaagcggggccgcaagaagctgctgtatattttcaaacagccctttatgcgccctgtgcagaccacccaggaagaggacggctgctcctgcaggttcccagaagaggaagagggagggtgcgagctgagggtgaaattctccagaagcgccgatgcacccgcatatcagcagggacagaaccagctgtacaacgagctgaatctgggacggagagaggagtatgatgtgctggataagcgcaggggcagagaccccgagatgggcggcaaacctagacgcaagaaccctcaggagggcctgtataatgagctgcagaaggataagatggctgaagcctattctgagatcggaatgaagggtgagagaaggcggggcaaagggcacgatggactgtaccagggactgtctactgctactaaggacacctacgacgccctgcatatgcaggccctgcccccccgcgcgcgcgagacgggaagcggagctactaacttcagcctgctgaagcaggctggcgacgtggaggagaaccctggacctggtctccggccggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcatcgataccgtcgacctcgatcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 11 - DNA, 4392 bp, synthetic construct
  source 1..4392: mol_type = other DNA
gtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctacggcggcggcggagtttggccgtaggtggcgccctcttcctcccatgcgtgtgaccccgaagcccctcatcggctgaagcagggccaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagcccttgagtcaaagacgtagtcgttgcaagtccgcaccaggtactgatatcccaccaaaaagtgcggcggcggctggcggtagaggggccagcgtagggtggccggggctccgggggcgaggtcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtgaggcgtgcgcagtcgttgacgctctagaccgtgcaaaaggagagcctgtaagcgggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttggcttccttccaggcgcggcggctgctgcgctagcttttttggccactggccgcgcgcggcgtaagcggttaggctggaaagcgaaagcattaagtggctcgctccctgtagccggagggttattttccaagggttgagtcgcaggacccccggttcgagtctcgggccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctccccttctcctaccgcgtcaggaggggcaacatccgcggctgacgcggcggcagatggtgattacgaacccccgcggcgccgggcccggcactacctggacttggaggagggcgagggcctggcgcggctaggagcgccctctcctgagcgacacccaagggtgcagctgaagcgtgacacgcgcgaggcgtacgtgccgcggcagaacctgtttcgcgaccgcgagggagaggagcccgaggagatgcgggatcgaaagttccacgcagggcgcgagttgcggcatggcctgaaccgcgagcggttgctgcgcgaggaggactttgagcccgacgcgcggaccgggattagtcccgcgcgcgcacacgtggcggccgccgacctggtaaccgcgtacgagcagacggtgaaccaggagattaactttcaaaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctaga

SEQ ID NO: 12 - DNA, 5855 bp, synthetic construct
  source 1..5855: mol_type = other DNA
cacatttccccgaaaagtgccacctgacgtggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgccaccatgggctttactaccaagatcatcttcctgtacaacctggtgctggtgtacgccggcttcgacgatcctagaaaggccatcgagctggtgcagaagagatacggcagaccctgcgattgctctggcggccaagtttctgagcctcctagcgacagagtgtcccaagtgacctgtagcggcaagaccgcctacctgatgcctgaccagagatggaagtgcaagagcatccccaaggacacaagccctagcggccctctgcaagagtgcccctgtaatagctaccagagcagcgtgcacagctcctgctacacaagctaccagcagtgcagaagcggcaacaagacctactacaccgccacactgctgaaaacccagaccggcggcacatccgatgttcaggtgctgggcagcaccaacaagctgattcagagcccctgcaacggcatcaagggccagagcatctgctggtccacaacagcccctatccacgtgtcagatggcggcggacctctggacaccaccagaatcaagagcgtgcagcggaagctggaagagatccacaaggctctgtaccccgagctgcagtatcaccctctggctatccccaaagtgcgggacaacctgatggtggacgcccagacactgaatatcctgaacgccacctacaatctgctgctgatgagcaacaccagcctggtggacgattgctggctgtgcctgaaactgggccctcctacacctctggccattcctaactttctgctgagctacgtgacccggtccagcgacaatatcagctgcctgatcatcccacctctgctcgtgcagcccatgcagttcagcaactcctcctgcctgttcagccccagctacaacagcaccgaggaaatcgatctgggacacgtggccttcagcaattgcaccagcatcaccaacgtgacaggccccatctgtgccgtgaatggcagcgtgttcctgtgtggcaacaacatggcctacacctacctgcctaccaactggaccggactgtgtgtgctggctaccctgctgccagacatcgacattatccctggcgacgagcccgtgcctattcctgccatcgaccacttcatctacagacccaagcgggctatccagttcatccctctgctggccggactgggaatcaccgccgcttttacaacaggcgctacaggcctgggagtgtctgtgacccagtacaccaagctgagcaaccagctgatctccgacgtgcagatcctgagcagcaccatccaggacctgcaggatcaggtggactctctggctgaagtggtgctgcagaacagaagaggcctggatctgctgacagccgagcaaggcggaatttgtctggccctgcaagagaagtgctgcttctacgtgaacaagagcggcatcgtgcgcgacaagatcaagacactgcaagaggaactggaacggcggagaaaggacctggcctccaatcctctgtggacaggactgcaaggcctgctgccttatctgctgccatttctgggcccactgctgacattgctgttgctgctgacaatcggcccctgcatcttcaacagactggtgcagttcgtgaaggaccggatctctgtggtgcaggctctggtgctgacacagcagtaccaccagctgaagcccctggaatacgagccctaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcg

SEQ ID NO: 13 - DNA, 12150 bp, synthetic construct
  source 1..12150: mol_type = other DNA
ttgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcgatcccgtaataagcttcgaggtccgcggccggccgcgttgacgcgcacggcaagaggcgaggggcggcgactggtgagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagctcgaggccgccccggtgaccttcagaccttggcactggaggtggcccggcagaagcgcggcatcgtggatcagtgctgcaccagcatctgctctctctaccaactggagaactactgcaactaggcccaccactaccctgtccacccctctgcaatgaataaaacctttgaaagagcactacaagttgtgtgtacatgcgtgcatgtgcatatgtggtgcggggggaacatgagtggggctggctggagtggcgatgataagctgtcaaacatgagaattaattcttgaagacgaaagggcctcgtgatacgcctatttttataggttaatgtcatgataataatggtttcttagtctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaacccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttgggctgcaggaattaattcgagctcgcccgaca

(19) United States
(12) Patent Application Publication
(10) Pub. No.: US 2026/0000711 A1
(43) Pub. Date: Jan. 1, 2026

(54) B7-H3 TARGETED SINGLE-DOMAIN ANTIBODY, CHIMERIC ANTIGEN RECEPTOR MOLECULE, CHIMERIC ANTIGEN RECDPTOR CELL, AND USE THEREOF IN PREPARATION OF DRUGS FOR TREATING NASOPHARYNGRAL CARCINOMA

(71) Applicant: Shenzhen Houpu Medical and Health Technology Co., Ltd., Shenzhen (CN)

(72) Inventors: JUN WANG, Shenzhen (CN); HAIWEN CHEN, Shenzhen (CN)

(21) Appl. No.: 19/204,604

(22) Filed: May 11, 2025

(30) Foreign Application Priority Data
Jun. 12, 2024 (CN) 202410751513X

(51) Int. Cl. A61K 35/17; A61K 40/11; A61K 40/31; A61K 40/42; C07K 14/705; C07K 14/725; C07K 16/28; C12N 15/86

(52) U.S. Cl. CPC A61K 35/17; A61K 40/11; A61K 40/31; A61K 40/421; C07K 14/7051; C07K 14/70517; C07K 14/70578; C07K 16/2827; C12N 15/86; A61K 2239/13; A61K 2239/17; A61K 2239/21; C07K 2317/53; C07K 2317/569; C07K 2319/02; C07K 2319/03; C12N 2740/16043

(57) ABSTRACT

The present disclosure provides a targeted B7-H3 single-domain antibody, a chimeric antigen receptor molecule, a chimeric antigen receptor cell, and their application in the preparation of drugs for treating nasopharyngeal carcinoma, which belongs to the field of antibody drug technology; an amino acid sequence of the B7-H3 targeted single-domain antibody provided by the present disclosure is shown in SEQ ID No. 1, and an amino acid sequence of the chimeric antigen receptor molecule is shown in SEQ ID No. 2; the chimeric antigen receptor cell provided by the present disclosure can specifically bind to tumor cells through targeting B7-H3 and exert specific antitumor effects via antigen-dependent mechanism, making it one of the potential effective methods for treating recurrent and metastatic NPC; according to the records of the embodiments, after co-culturing the chimeric antigen receptor cell provided by the present disclosure with the nasopharyngeal carcinoma cell line HK1-EBV, it can significantly kill nasopharyngeal carcinoma cell.

### REFERENCE TO AN ELECTRONIC SEQUENCE LISTING

The sequence listing of the present disclosure is submitted electronically via EFS-Web. The contents of the electronic sequence listing (Name: SequenceListing.xml; Size: 53,137 bytes; and Date of Creation: Sep. 5, 2025) is herein incorporated by reference in its entirety.

### CROSS-REFERENCE TO RELATED APPLICATIONS

This application claims the benefit of priority from Chinese patent application No. 202410751513.X, filed on Jun. 12, 2024. The content of the aforementioned application, including any intervening amendments thereto, is incorporated herein by reference in its entirety.

### FIELD

The present disclosure relates to the field of antibody drug technology, and particularly relates to B7-H3 targeted single-domain antibody, chimeric antigen receptor molecule, chimeric antigen receptor cell, and its use in the preparation of drugs for treating nasopharyngeal carcinoma.

### BACKGROUND

With the advancements in tumor immunotherapy and clinical biotechnology, chimeric antigen receptor T cell (CAR-T) immunotherapy has currently emerged as one of the most promising cancer immunotherapies. The chimeric antigen receptor (CAR) consists of three domains: the extracellular antigen-binding domain, the transmembrane domain, and the intracellular signal transduction domain. The extracellular antigen-binding domain includes a single-chain fragment variable (scFv) that targets tumor antigens. Through the genetic transfection technology, CAR molecules are expressed on the surfaces of T cells, which are subsequently co-cultured with tumor cells. After CAR molecules bind to the tumor antigens, CAR molecules activate T cells and exert specific antitumor effects through antigen-dependent mechanism, without being restricted by major histocompatibility complex (MHC).

The single-domain antibody (sdAb) is a type of antibody that lacks the light chain and only has the variable region of the heavy chain. Due to its diminutive molecular weight, it is also known as Nanobody. Camelid animals produce sdAb with only the heavy chains, where the heavy chain antigen-binding fragment contains the variable domain of heavy chain of heavy-chain antibody (VHH). VHH can maintain high affinity for antigen even without the light chain.

Nasopharyngeal carcinoma (NPC), a malignant epithelial tumor predominantly arising in the nasopharynx, is often accompanied by Epstein-Barr (EB) virus infection and has a regional clustering pattern. It is one of the most common malignant tumors. Clinically, NPC may present with bloody nasal discharge, tinnitus, nasal congestion, headache, decreased vision, and cranial nerve damage. The etiology of NPC has not yet been fully elucidated, but it is thought to be closely related to EB virus infection. Most NPC patients are sensitive to radiotherapy, and early-stage NPC patients are mainly treated with radiation therapy alone. For locally advanced patients, a combination of radiotherapy and chemotherapy is considered. However, there is currently a lack of effective means for advanced patients and recurrence patients after treatment.

### SUMMARY

In view of this, the present disclosure aims to provide a B7-H3 targeted single-domain antibody, a chimeric antigen receptor molecule, a chimeric antigen receptor cell, and the use in preparation of drugs for treating nasopharyngeal carcinoma; EB virus infection promotes NPC cells to upregulate B7-H3 expression, while B7-H3 is lowly expressed in normal tissues. The B7-H3 targeted single-domain antibody, the chimeric antigen receptor molecule, or the chimeric antigen receptor cell provided by the present disclosure can specifically bind to tumor cells by targeting B7-H3 and exert specific antitumor effects through antigen-dependent mechanism, making it one of the potential effective methods for treating recurrent and metastatic NPC.

The present disclosure provides a B7-H3 targeted single-domain antibody, an amino acid sequence of the single-domain antibody is shown in SEQ ID NO. 1.

The present disclosure provides a chimeric antigen receptor molecule (SEQ ID NO. 3), including a CD8a signal peptide (encoded by SEQ ID NO. 4), the single-domain antibody, a CD8a hinge domain (SEQ ID NO. 5), a CD8a transmembrane domain (SEQ ID NO. 6), a 41BB costimulatory domain (SEQ ID NO. 7), and a CD3z signal transduction domain (SEQ ID NO. 8) which are connected in sequence.

Preferably, an amino acid sequence of the chimeric antigen receptor molecule is shown in SEQ ID NO. 2.

The present disclosure provides a B7-H3 targeted lentiviral vector, a construction method includes:
1) cloning DNA sequence configured for encoding the chimeric antigen receptor molecule into a pHIV-EGFP vector (SEQ ID NO. 9), to obtain a recombinant expression plasmid pHIV-B7-H3-EGFP (SEQ ID NO. 10);
2) transfecting the recombinant expression plasmid pHIV-B7-H3-EGFP obtained in step 1), packaging plasmids pAdVntage (SEQ ID NO. 11), pBabTR (SEQ ID NO. 12) and pCMV-dR8.91 (SEQ ID NO. 13) into a cell at a mass ratio of about (4.5˜5.5):(2.5˜3.5): 1:(0.4˜0.6) for cultivation; and
3) collecting supernatant after cultivation, to obtain the B7-H3 targeted lentiviral vector.

Preferably, after step 3), the method further includes: mixing the supernatant with PEG 8000, centrifuging, collecting precipitate, and resuspending the precipitate, to obtain a concentrated B7-H3 targeted lentiviral vector.

Preferably, a volume ratio of the supernatant to PEG 8000 is about 1:(4˜6).

Preferably, the operation of “resuspending the precipitate” is carried out using a PBS solution, and a volume ratio of the precipitate to the PBS solution is about 1:(80˜120).

The present disclosure provides a B7-H3 targeted chimeric antigen receptor cell, which expresses the chimeric antigen receptor molecule.

The present disclosure provides a method for preparing the B7-H3 targeted chimeric antigen receptor cell, which includes: transplanting the lentiviral vector into PBMCs cell, to obtain the B7-H3 targeted chimeric antigen receptor cell.

The present disclosure provides a use of the B7-H3 targeted single-domain antibody, the chimeric antigen receptor molecule, the B7-H3 targeted chimeric antigen receptor cell, or the lentiviral vector in a preparation of drugs for treating nasopharyngeal carcinoma.

The present disclosure further provides a use of the B7-H3 targeted single-domain antibody, the chimeric antigen receptor molecule, the B7-H3 targeted chimeric antigen receptor cell, or the lentiviral vector in a preparation of reagents for killing nasopharyngeal carcinoma cell.

Preferably, the nasopharyngeal carcinoma cell is HK1-EBV cell line.

Compared with existing technologies, the present disclosure has the following beneficial effects: the B7-H3 targeted single-domain antibody, the chimeric antigen receptor molecule, or the chimeric antigen receptor cell provided by the present disclosure can specifically bind to tumor cell through targeting B7-H3, and exert specific antitumor effects via the antigen-dependent mechanism. It represents one of the potential effective methods for treating recurrent and metastatic NPC; according to the records in the embodiments, after co-culturing the chimeric antigen receptor cell with the nasopharyngeal carcinoma cell line HK1-EBV provided by the present disclosure, they can significantly kill nasopharyngeal carcinoma cell.

### BRIEF DESCRIPTION OF THE DRAWINGS

FIG. 1 is a schematic diagram of the molecular structure of the B7-H3 targeted chimeric antigen receptor molecule;

FIG. 2 is the structural map of the expression plasmid of pHIV-B7-H3-EGFP;

FIG. 3 is the structural map of pBabTR plasmid;

FIG. 4 is the positive rate of the B7-H3 targeted chimeric antigen receptor cell; and

FIG. 5 is the results of in vitro killing of nasopharyngeal carcinoma cells by B7-H3 targeted chimeric antigen receptor cell, where the left figure is the control group and the right figure is the B7-H3 CAR-T treated group.

### DETAILED DESCRIPTION

The present disclosure provides a B7-H3 targeted single-domain antibody, the amino acid sequence of which is shown in SEQ ID No. 1, as follows:

EVQLVESGGGLVQPGGSLRLSCAASGFPFSNYAMSWYRQAPGKERE
LVANIGGPSGYSTYYKEAVKG RFTISRDNAKTTVYLHMNSLKPDDTAVYF
CKAVARIGVPQEYWGQGTQVTVSS.

The present disclosure provides a chimeric antigen receptor molecule, including a CD8a signal peptide, the single-domain antibody, a CD8a hinge domain, a CD8a transmembrane domain, a 41BB co-stimulation domain and a CD3z signal transduction domain, which are connected in sequence.

In the present disclosure, the amino acid sequence of the chimeric antigen receptor molecule is shown in SEQ ID No. 2 and is specifically as follows:

MALPVTALLLPLALLLHAARPEVQLVESGGGLVQPGGSLRLSCAASG
FPFSNYAMSWYRQAPGKERE
LVANIGGPSGYSTYYKEAVKGRFTISRDNAKTTVYLHMNSLKPDDTAVYFCKAVA
RIGVPQEYWGQGTQVTVSSTT
TPAPRPPTPAPTIASQPLSLRPEACRPAAGGAVHTRGLDFACDIYIWAPLAGTCGVL
LLSLVITLYCNKRGRKKLL
YIFKQPFMRPVQTTQEEDGCSCRFPEEEEGGCELRVKFSRSADAPAYQQGQNQLY
NELNLGRREEYDVLDKRRGRD
PEMGGKPRRKNPQEGLYNELQKDKMAEAYSEIGMKGERRRGKGHDGLYQGLST
ATKDTYDALHMQALPPR.

The present disclosure provides a B7-H3 targeted chimeric antigen receptor cell, which expresses the chimeric antigen receptor molecule. In the present disclosure, the B7-H3 targeted chimeric antigen receptor cell is preferably obtained by transfecting a B7-H3 targeted lentiviral vector into PBMCs stimulated with CD3/CD28 and then culturing them.

The present disclosure provides a B7-H3 targeted lentiviral vector, and a construction method includes the following steps:
1) cloning DNA sequence configured for encoding the chimeric antigen receptor molecule into a pHIV-EGFP vector, to obtain a recombinant expression plasmid pHIV-B7-H3-EGFP;
2) transfecting the recombinant expression plasmid pHIV-B7-H3-EGFP obtained in step 1), packaging plasmids pAdVntage, pBabTR and pCMV-dR8.91 into cell at a ratio of about (4.5˜5.5):(2.5˜3.5): 1:(0.4˜0.6) for cultivation; and
3) collecting supernatant after cultivation to obtain the B7-H3 targeted lentiviral vector.

In the present disclosure, the DNA sequence configured for encoding the chimeric antigen receptor molecule is cloned into the pHIV-EGFP vector to obtain the recombinant expression plasmid pHIV-B7-H3-EGFP; in the present disclosure, the pHIV-EGFP vector is preferably purchased from Shanghai HeWu Biotechnology Co., Ltd., catalog No. P25226; the DNA sequence configured for encoding the chimeric antigen receptor molecule is synthesized by a biotech company and then cloned into the pHIV-EGFP vector; the above operations are preferably carried out by the biotech company.

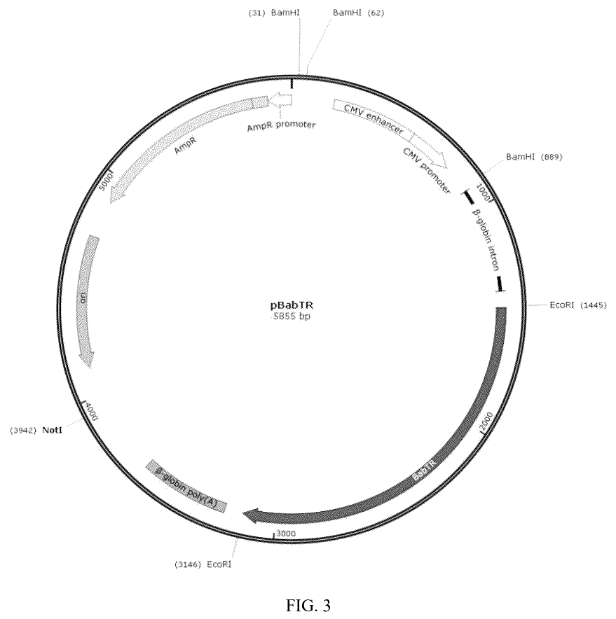

After obtaining the recombinant expression plasmid pHIV-B7-H3-EGFP, the recombinant expression plasmid pHIV-B7-H3-EGFP, packaging plasmids pAdVntage, pBabTR, and pCMV-dR8.91 are transfected into cell at a mass ratio of about (4.5˜5.5):(2.5˜3.5): 1:(0.4˜0.6) for cultivation. In the present disclosure, the mass ratio of the recombinant expression plasmid pHIV-B7-H3-EGFP, packaging plasmids pAdVntage, pBabTR, and pCMV-dR8.91 is preferably about (4.8˜5.2):(2.8˜3.2): 1:(0.45˜0.55), more preferably 5:3:1:0.5. In the present disclosure, the packaging plasmids pAdVntage and pCMV-dR8.91 are preferably purchased from Shanghai Hewu Biotechnology Co., Ltd.; the pBabTR plasmid is synthesized by Beijing Ruibo Xingke Biotechnology Co., Ltd., and the structural map of the pBabTR plasmid is shown in FIG. 3. In the present disclosure, the cell is preferably 293T cell, which is preferably purchased from the American Type Culture Collection ATCC. In the present disclosure, the cultivation time is preferably 48˜72 h. In the present disclosure, the supernatant is preferably collected at 48 h and 72 h of cultivation, and the collection is preferably performed by filtering with a membrane, a pore size of the membrane is preferably 0.45 μm. After collecting the supernatant, the method preferably includes: mixing the supernatant with PEG 8000, centrifuging, collecting precipitate, and resuspending the precipitate to obtain a concentrated B7-H3 targeted lentiviral vector. In the present disclosure, a volume ratio of the supernatant to PEG 8000 is preferably about 1:(4˜6), more preferably 1:5, with the PEG 8000 preferably purchased from Sigma-Aldrich, catalog No. 89510; in the present disclosure, a temperature for centrifugation is preferably about 3˜5° C., more preferably 4° C.; a centrifugal force is preferably about 3500˜4500 g, more preferably 3800˜4200 g, even more preferably 4000 g; a time for centrifugation is preferably about 40˜50 min, more preferably 42˜48 min, most preferably 45 min. After centrifugation, the precipitate is collected and resuspended in PBS solution, with the ratio of the precipitate to PBS solution being preferably about 1:(80˜120), more preferably 1:(90˜110), even more preferably 1:100. In the present disclosure, the concentrated B7-H3 targeted lentiviral vector is preferably stored at a low temperature, more preferably at −80° C.

The present disclosure provides a B7-H3 targeted chimeric antigen receptor cell which expresses the chimeric antigen receptor molecule.

The present disclosure also provides a method for preparing the B7-H3 targeted chimeric antigen receptor cell, which includes the following steps: transfecting PBMCs cell with the lentiviral vector to obtain the B7-H3 targeted chimeric antigen receptor cell. The specific transfection procedures of the present disclosure are not particularly limited; conventional transfection methods in the field can be used.

The present disclosure also provides the use of the B7-H3 targeted single-domain antibody, the chimeric antigen receptor molecule, the B7-H3 targeted chimeric antigen receptor cell, or the lentiviral vector in the treating for nasopharyngeal carcinoma. In the present disclosure, the B7-H3 targeted chimeric antigen receptor cell can significantly kill the nasopharyngeal carcinoma cell line HK1-EBV and can be used as drugs to inhibit the nasopharyngeal carcinoma cell line HK1-EBV or nasopharyngeal carcinoma.

The technical solutions provided by the present disclosure are described in detail below with reference to the embodiments, but the embodiments should not be understood as limiting the protection scope of the present disclosure.

### Embodiment 1

The B7-H3 targeted chimeric antigen receptor molecule is designed.

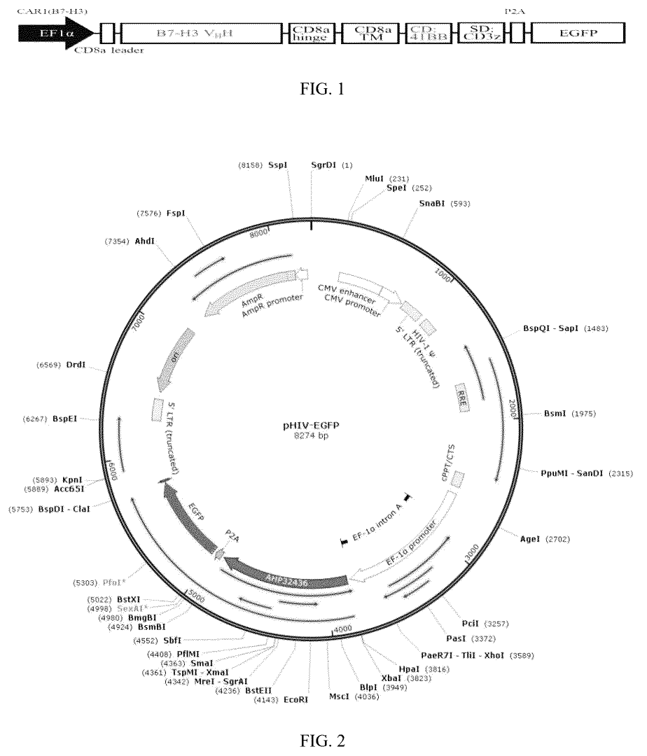

The camel monoclonal antibody (shown in SEQ ID No. 1) selected for B7-H3 are linked to CD8a signal peptide, hinge domain and transmembrane domain, 41BB co-stimulation domain and CD3z signal transduction domain, respectively. The structure of the B7-H3 targeted chimeric antigen receptor molecule is shown in FIG. 1, with the amino acid sequence provided in SEQ ID No. 2.

The B7-H3 targeted chimeric antigen receptor CAR-T cell is constructed.

The DNA sequence used for encoding the B7-H3 chimeric antigen receptor molecule is constructed to the pHIV-EGFP vector to form the pHIV-B7-H3-EGFP expression plasmid (which is entrusted to Biological Company), and the plasmid map is shown in FIG. 2.

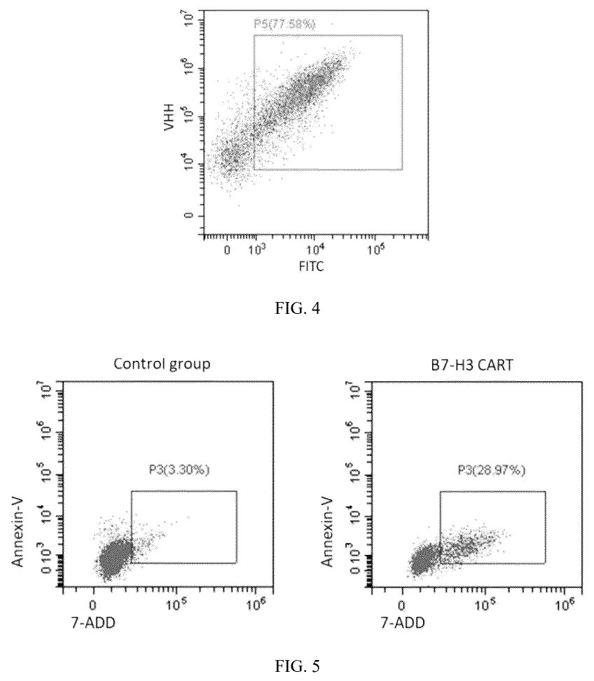

An empty plasmid or the successfully constructed target expression plasmid is transfected into 293T cell (purchased from American Type Culture Collection Center ATCC) with the packaging plasmid pAdVntage (purchased from Shanghai Hewu Biotechnology Co., Ltd., Catalog No.: P2356), pBabTR (purchased from Beijing Ruibo Xingke Biotechnology Co., Ltd., the plasmid map is shown in FIG. 3), and pCMV-dR8.91 (purchased from Shanghai Hewu Biotechnology Co., Ltd., Catalog No.: P3110) at a ratio of 5:3:1:0.5. The supernatant is collected at 48 h and 72 h of cultivation and filtered through a 0.45 μm filter, and PEG 8000 (purchased from Sigma-Aldrich, catalog No.: 89510) is added into the supernatant. The supernatant is centrifuged at 4° C. for 45 min at 4000 g after thorough mixing, the supernatant is discarded, the precipitate is resuspended in PBS at a ratio of 1:100, and the precipitate is stored at −80° C. for later use. PBMCs from healthy individuals stimulated with CD3/CD28 is infected with concentrated virus; a volume ratio of concentrated virus to PBMCs is 50 μl:500 μl with a concentration of 106 cells/ml. In vitro, 500 IU/ml IL-2 was added to maintain the growth of infected PBMCs. After culturing for 20 days, VHH flow cytometry is used to detect CAR molecule expression, shown in FIG. 4, with a positive rate of approximately 77%.

### Embodiment 2

The B7-H3 CAR-T cell prepared in Embodiment 1 is mixed with the nasopharyngeal carcinoma cell line HK1-EBV (from the Cancer Prevention and Treatment Center of Sun Yat-sen University) at a ratio of about 5:1 for co-culture for 6 hours. The tumor cell is then labeled with flow cytometry antibodies 7-AAD and ANNEXIN-V, and apoptosis is detected using flow cytometry. A control group is set up, where T cell transfected with an empty vector is co-cultured with the nasopharyngeal carcinoma cell line HK1-EBV under identical conditions to the experimental group. Compared to the control group, B7-H3 CAR-T cell significantly kill the tumor cells, shown in FIG. 5.

The above description is merely the preferred embodiment of the present disclosure. It should be noted that, for those skilled in the art, various modifications and improvements can be made without departing from the principles of the present disclosure, which also fall within the protection scope of the present disclosure.

What is claimed is:

1. A B7-H3 targeted single-domain antibody, wherein an amino acid sequence of the single-domain antibody is shown in SEQ ID NO. 1.

2. A chimeric antigen receptor molecule, comprising a CD8a signal peptide, the single-domain antibody of claim 1, a CD8a hinge domain, a CD8a transmembrane domain, a 41BB costimulatory domain, and a CD3z signal transduction domain which are connected in sequence.

3. The chimeric antigen receptor molecule according to claim 2, wherein an amino acid sequence of the chimeric antigen receptor molecule is shown in SEQ ID NO. 2.

4. A B7-H3 targeted lentiviral vector, wherein a construction method comprises:
1) cloning DNA sequence configured for encoding the chimeric antigen receptor molecule of claim 3 into a pHIV-EGFP vector to obtain a recombinant expression plasmid pHIV-B7-H3-EGFP;
2) transfecting the recombinant expression plasmid pHIV-B7-H3-EGFP obtained in step 1), packaging plasmids pAdVntage, pBabTR and pCMV-dR8.91 into a cell at a mass ratio of about (4.5˜5.5):(2.5˜3.5): 1:(0.4˜0.6) for cultivation; and
3) collecting supernatant after cultivation to obtain the B7-H3 targeted lentiviral vector.

5. The lentiviral vector according to claim 4, wherein after step 3), the method further comprises: mixing the supernatant with PEG 8000, centrifuging, collecting precipitate, and resuspending the precipitate, to obtain a concentrated B7-H3 targeted lentiviral vector.

6. The lentiviral vector according to claim 5, wherein a volume ratio of the supernatant to PEG 8000 is about 1:(4˜6).

7. The lentiviral vector according to claim 5 or 6, wherein the operation of “resuspending the precipitate” is carried out using a PBS solution, and a volume ratio of the precipitate to the PBS solution is about 1:(80˜120).

8. A B7-H3 targeted chimeric antigen receptor cell, wherein the chimeric antigen receptor cell expresses the chimeric antigen receptor molecule of claim 2 or 3.

9. A method for preparing the B7-H3 targeted chimeric antigen receptor cell of claim 8, comprising:
transplanting the lentiviral vector according to any one of claims 4 to 7 into PBMCs cell to obtain the B7-H3 targeted chimeric antigen receptor cell.

10. A use of the B7-H3 targeted single-domain antibody of claim 1, the chimeric antigen receptor molecule of claim 2 or 3, the lentiviral vector of any one of claims 4 to 7, and the B7-H3 targeted chimeric antigen receptor cell of claim 8 in a preparation of drugs for treating nasopharyngeal carcinoma.
